(19) United States
(12) Patent Application Publication
(10) Pub. No.: US 2020/0138100 A1
(43) Pub. Date: May 7, 2020

(54) DUAL-PURPOSE SMOKING DEVICE AND HYDROGEN INHALER

(71) Applicant: Aqua Bank CO.,LTD., Osaka-shi, Osaka (JP)

(72) Inventor: Takashi TAKEHARA, Osaka-shi, Osaka (JP)

(21) Appl. No.: 16/607,474

(22) PCT Filed: Oct. 19, 2018
(86) PCT No.: PCT/JP2018/039065

§ 371 Date: Oct. 23, 2019

(30) Foreign Application Priority Data
Nov. 8, 2017 (JP) 2017-215875
Dec. 15, 2017 (JP) 2017-241100

(51) Int. Cl. A24F 40/40; A24F 40/20; A24F 40/30; A24F 40/57; A24F 40/51; C25B 1/04; H01M 16/00; A61M 15/06; A61M 15/02

(52) U.S. Cl. CPC A24F 40/40; A61M 2205/587; A24F 40/30; A24F 40/57; A24F 40/51; C25B 1/04; H01M 16/00; A61M 15/06; A61M 15/02; H01M 2220/30; A61M 2205/8206; A61M 2202/02; A61M 2205/3331; A61M 2205/36; A24F 40/20

(57) ABSTRACT

A smoking/hydrogen-suction device detachably accommodates a tubular portable heating-type smoking device main body and makes it possible to perform smoking and hydrogen suction. The smoking/hydrogen-suction device includes: a pair of positive/negative electrodes that are electrically conducted to or cut off from a positive electrode and a negative electrode of a battery by a control substrate; an electrolysis tank which is capable of storing water, into a lower portion of which the pair of positive/negative electrodes are inserted, and which has an injection port for an electrolyte or water and a hydrogen generation port fluidically connected to an air layer in the electrolysis tank; and a smoking device receiving portion having a tubular hole into which the smoking device main body can be inserted from above and having at its bottom portion a terminal that can be electrically connected to a terminal at a lower portion of the smoking device.

### TECHNICAL FIELD

The present invention relates to a smoking/hydrogen-suction device configured to be connected to a smoking device main body of a general-purpose portable heating-type smoking device to allow suction of nicotine or an aromatic gas and, at the same time, allowing suction of hydrogen gas generated through electrolysis.

### BACKGROUND ART

An evil of passive smoking due to sidestream smoke generated during smoking and due to exposure to tobacco smoke has become a social issue, and there has been provided a so-called electronic cigarette which emits no sidestream smoke containing tar, etc. or in which the amount of sidestream smoke, etc. is reduced. In recent years, as the electronic cigarette, there exists a type in which a throwaway cartridge containing tobacco leaf is connected and used each time smoking is performed and in which tobacco leaf in the throwaway cartridge is heated without being ignited so that solely the nicotine ingredient may be sucked in, with the sidestream smoke being prevented from being emitted (or being substantially reduced) (Patent Literature 1 through 3).

In this heating-type electronic cigarette, a tubular main body equipped with a heater configured to heat by being electrically conducted, a tubular power supply cartridge (including a rechargeable battery) connected to the lower portion of the main body and configured to supply power to the main body, and a throwaway smoking cartridge which is attached to a hollow steam chamber provided in the upper/inner portion of the main body and which generates a steam containing nicotine when heated are combined with each other to constitute an electronic cigarette.

In the case of the conventional electronic cigarette, however, while it is possible to avoid the harmfulness to the health of passive smoking due to the sidestream smoke, etc., at least the smoker himself continues to take in nicotine. It is indispensable for him to gradually reduce the nicotine intake amount of his own accord to quit smoking. After all, this does not help to quit smoking.

Conventionally, as the harmful effects of tobacco to the health, the following have been pointed out: (1) an increase in the risk of development of cancer due to the generation of active oxygen; and (2) an increase in the risk of motor disturbance or hardening of the arteries due to inhaling of carbon monoxide. Regarding item (1), active oxygen exhibits a very strong oxidizing force and serves to remove bacteria and viruses having entered the human body, whereas, as is known, it also attacks and damages normal human cells. Excessive intake of active oxygen increases the possibility of damaging normal cells, and leads to the risk of deterioration and mutation of the cells and skin aging. Typically, it increases the risk of developing cancer.

Regarding item (2), the intake of carbon monoxide, even in a small amount, leads to deterioration in the function of blood hemoglobin, and a slight oxygen shortage continues, which leads to deterioration in motor function and brain function. In some cases, the red blood cells increase to an abnormal degree, resulting, as pointed out, in high blood viscosity, an increase in blood pressure, and headache, leading sometimes to hardening of the arteries.

In this regard, in recent years, it has become apparent that hydrogen has a high function to remove active oxygen since hydrogen and active oxygen are combined to be reduced. Further, as is already known, the resultant substance of reaction between hydrogen and active oxygen is water, which is of very little negative influence on the human body. Thus, it is recommended that hydrogen be taken into the body during smoking, which is particularly likely to generate active oxygen in the body, which has been one of the causes in the diversion of the market of hydrogen water. Further, when combined with oxygen, carbon monoxide is turned into carbon dioxide to be detoxified, so that, during smoking, it is also recommended to take in oxygen.

In view of the above-mentioned circumstances, the present applicant has provided a portable smoking/hydrogen-suction device which allows intake of nicotine and hydrogen, etc. and which makes it possible to simultaneously attain prevention of passive smoking and a reduction in the harmfulness to the health of the smoker himself.

### CITATION LIST

### Patent Literature

Patent Literature 1: JP 2017-500100 T

Patent Literature 2: JP 2016-510970 T

Patent Literature 3: JP 2014-524313 T

### SUMMARY OF INVENTION

### Technical Problem

However, the user must purchase the above-described portable smoking/hydrogen-suction device separately from the electronic cigarette that he/she already possesses and carry it about, so that in some cases, as has been pointed out, the user's appetite for buying the device is sapped when the cost and the inconvenience of carrying it about are taken into account.

The present invention has been made in view of the above circumstances. It is an object of the present invention to provide a portable smoking/hydrogen-suction device making it possible to take in nicotine and hydrogen (or an aromatic gas) while using a general-purpose electronic cigarette.

### Solution to Problem

The present invention provides a smoking/hydrogen-suction device which detachably accommodates a tubular portable heating-type smoking device main body and which makes it possible to smoke and suck in hydrogen, including:

a rechargeable battery arranged in a battery receiving portion;

a control substrate controlling power supply from the battery;

a pair of positive/negative electrodes that are electrically conducted to or cut off from a positive electrode and a negative electrode of the battery by the control substrate;

an electrolysis tank which is capable of storing water, into a lower portion of which the pair of positive/negative electrodes are inserted, and which has an injection port for an electrolyte or water and a hydrogen generation port fluidically connected to an air layer in an electrolysis tank; and

a smoking device receiving portion having a tubular hole into which the smoking device main body can be inserted from above and having at its bottom portion a terminal that can be electrically connected to a terminal at a lower portion of the smoking device,

wherein when power supply from the battery to the smoking device main body is effected by the control substrate, a smoking cartridge mounted to an upper portion of the smoking device is heated to generate a nicotine or aroma containing steam from the upper portion;

when power supply from the battery to the pair of positive/negative electrodes is effected by the control substrate, the electrolysis tank generates hydrogen from the positive electrode, and hydrogen is emitted from the hydrogen generation port; and

the smoking/hydrogen-suction device further comprises a lid member covering the hydrogen generation port of the electrolysis tank and the smoking cartridge and guiding the nicotine or aroma containing steam and the hydrogen respectively emitted from the smoking cartridge and the hydrogen generation port to a nozzle portion having a through hole.

The above-described smoking/hydrogen suction device has a specific structure utilizing a tubular portable heating-type smoking device main body as shown in Patent Literature 1 through 3 and attaining nicotine intake and hydrogen intake. The portable heating-type smoking device main body is a heating-type nicotine generation device detachably connected to the portion of the tubular portable heating-type smoking device which effects power supply, and a smoking cartridge containing tobacco leaf, which is an article of consumption, is attached to the upper portion thereof, and this is heated to emit a nicotine containing steam. This general-purpose portable heating-type smoking device main body is inserted into the smoking device receiving portion of the smoking device of the present invention, is connected (coupled) to the terminal at the lower portion, and receives power supply from the rechargeable battery in the device of the present invention to thereby emit a nicotine containing steam. In this sense, the present invention has the function of a substitute of the power supply portion of the general-purpose portable heating-type smoking device.

In the smoking/hydrogen-suction device of the present invention, hydrogen is generated through electrolysis of an electrolyte (or water) at the positive/negative electrodes in the electrolysis tank in the smoking/hydrogen-suction device. This electrolysis is effected through supply of power of the rechargeable battery by the control substrate. The nicotine containing steam and the hydrogen are guided to one nozzle portion by the lid member in the upper portion, and can be simultaneously sucked in. Thus, the user can take in a nicotine containing steam and hydrogen solely by sucking at one nozzle portion as in the case of smoking. When oxidized, hydrogen is reduced to water, so that it is desirable for hydrogen not to pass through heated air which is subject to oxidation. However, in the present smoking/hydrogen-suction device, hydrogen is guided to the nozzle portion by the lid portion, and advantageously does not pass the periphery of the main body being heated.

In electrolysis, hydrogen is generated at the positive electrode, whereas oxygen is generated at the negative electrode. In this sense, in the present smoking/hydrogen-suction device, it is also possible to perform suction of high concentration oxygen. At the upper portion of the electrolysis tank, there is provided an attachment having in the same member a water injection port capable of filling the electrolysis tank with water and a hydrogen generation port formed of a material solely allowing transmission of gas. Thus, it is possible to fill the tank with water from the outside and to emit solely the gas component such as hydrogen to the outside (into the lid member) without allowing water to leak out.

More specifically, the present smoking/hydrogen-suction device has a structure as described below.

The water injection port of the electrolysis tank is openable and closable, and the hydrogen generation port is shielded with a material solely allowing transmission of gas and not allowing transmission of liquid in the electrolysis tank, the water injection port and the hydrogen generation port being formed as an integral attachment.

In the state in which the smoking device main body is held in the smoking device receiving portion, the upper end of the smoking device main body and the attachment of the electrolysis tank are arranged in parallel, and the nozzle portion of the lid member covers the upper end of the smoking device in a telescopic fashion. In this state, the hydrogen guided from the hydrogen generation port of the attachment within the lid member is emitted to outside from the gap between the nozzle portion and the upper end of the smoking device.

When power supply is effected from the battery to the smoking device main body by the control substrate, the heater in the smoking device main body is heated, and the smoking cartridge containing a wet smokable substance is heated.

The smoking/hydrogen-suction device further includes, at the bottom portion of the smoking device receiving portion, a pressure sensor switch supplying power from the battery to the smoking device main body by the control substrate when the smoking device main body is inserted and downwardly depressed. It is desirable for the pressure sensor switch to have an ON/OFF switch. This is for the purpose of keeping the power source OFF in the case where steam containing nicotine, etc. is not to be sucked in even when the smoking device main body is in the accommodated state, thereby preventing steam emission from the smoking cartridge and consumption of the rechargeable battery.

The control substrate receives an electrical conduction signal to the pair of positive/negative electrodes based on an operation signal from the user to perform power supply from the battery to the pair of positive/negative electrodes based on each signal, and receives an electrical conduction signal to the smoking device main body based on a power supply signal from the pressure sensor switch to effect power supply from the battery to the smoking device main body based on the signal.

### Advantageous Effect of Invention

In the portable smoking/hydrogen-suction device of the present invention, it is possible to taken in nicotine and hydrogen (or an aromatic gas) with a single device while using a general-purpose electronic cigarette, making it possible to simultaneously attain prevention of passive smoking and a reduction in the harmfulness of the health of the smoker himself while taking in nicotine.

### BRIEF DESCRIPTION OF DRAWINGS

FIG. 1 is a block diagram schematically illustrating a smoking/hydrogen-suction device according to an embodiment of the present invention.

FIG. 2 is a front view, left and right side views, and a plan view of a smoking/hydrogen-suction device according to an embodiment of the present invention.

FIG. 3 is a schematic exploded view of a general-purpose electronic cigarette including the main body of the present smoking device.

FIG. 4 is a schematic diagram illustrating how electrolysis takes place in an electrolysis tank of the smoking/hydrogen-suction device of the present invention.

FIG. 5 is a schematic view of the specific structure of an electrode.

FIG. 6 is an external photographic view of a smoking/hydrogen-suction device according to an embodiment of the present invention.

### DESCRIPTION OF EMBODIMENTS

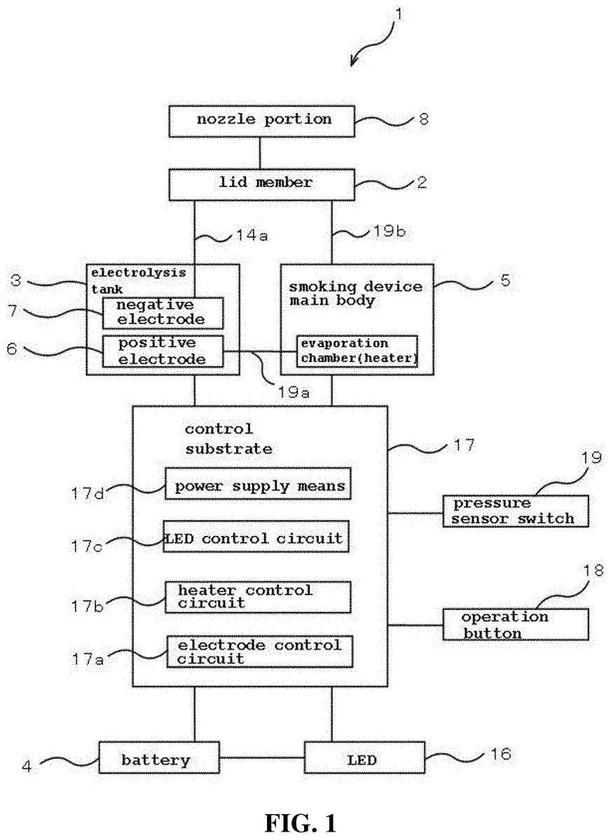

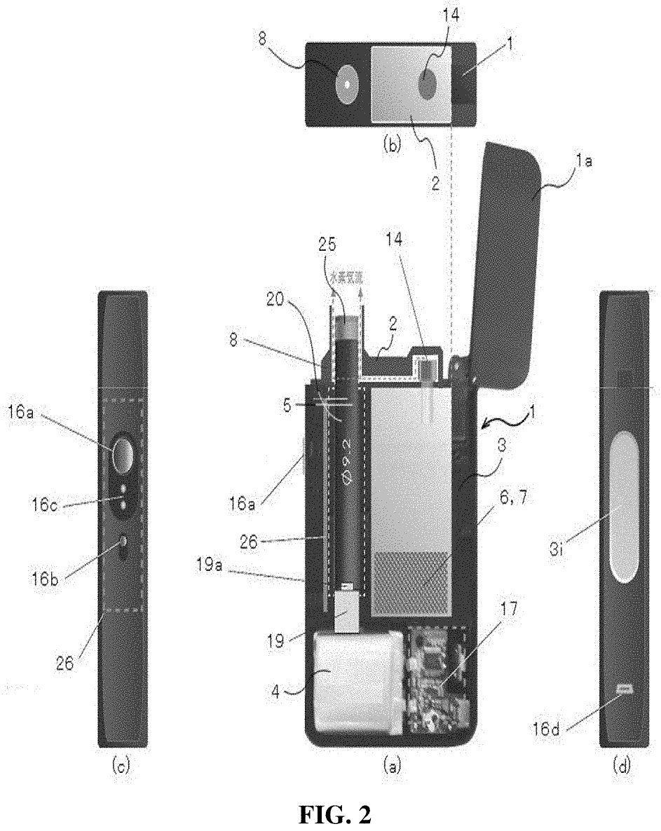

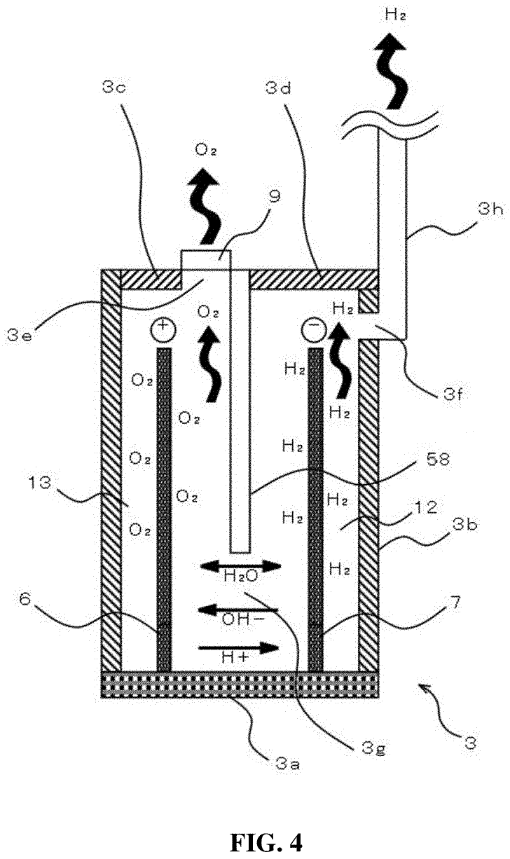

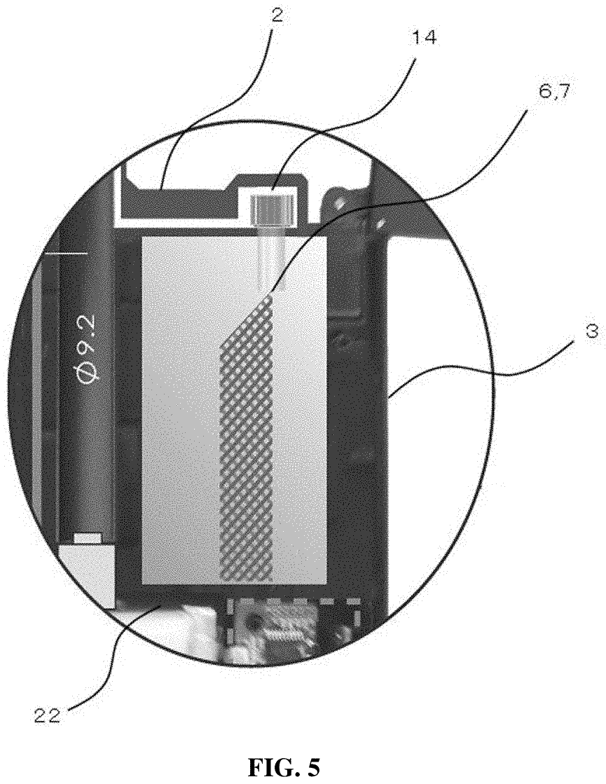

In the following, a smoking/hydrogen-suction device according to an embodiment of the present invention will be described. FIG. 1 is a block diagram illustrating the embodiment, FIG. 2 is six views, etc. of the embodiment of FIG. 1, and FIG. 4 is a schematic diagram illustrating how electrolysis takes place in an electrolysis tank of the smoking/hydrogen-suction device. FIG. 5 is a schematic diagram illustrating a specific structure of an electrode. It goes without saying that the smoking/hydrogen-suction device of the present invention is not restricted to the one shown in the drawings but includes modifications made without departing from the scope of what is shown and described from the viewpoint of common sense.

As shown in FIG. 1, the present smoking/hydrogen-suction device is generally composed of a battery 4, an LED 16, a control means 17, an electrolysis tank 3, a smoking device main body 5, a mixing portion 2, and a nozzle portion 8. First, the battery 4 is of the charging type, and a pair of positive/negative electrodes 6 and 7 are arranged in the electrolysis tank 3. Power from the battery 4 is supplied to the positive/negative electrodes 6 and 7 via a control means 33, and the LED 16 is connected to the battery 4. The control means 17 is equipped with an electrode control circuit 17a, a heater control circuit 17b, an LED control circuit 17c, and a power supply means (power supply circuit) 17d.

Further, a pressure sensor switch 19 is provided at the bottom portion of the receiving portion of the smoking device main body 5, and when the lower end of the smoking device main body depresses the pressure sensor switch 19, the power of the battery 4 is supplied to the smoking device main body 5 by the power supply means 17d of the control substrate 17.

When the user operates an operation button 18, the electrode control circuit 17a controls electrical conduction to and cutting off from the pair of electrodes 6 and 7 in the electrolysis tank 3, and the power is supplied to the electrodes 6 and 7, with the power amount supplied from the battery 4 being varied by the power supply means 17d. When power is supplied to the pair of electrodes 6 and 7, water stored in an electrolysis tank 10 undergoes electrolysis, and oxygen is generated at the positive electrode 6 side, and hydrogen is generated at the negative electrode 7 side.

The hydrogen generated from the negative electrode 7 flows into a lid member 2 via an attachment 14 at the upper portion of the electrolysis tank 3. The oxygen generated from the positive electrode 6 is vented.

In the smoking device main body 5, when the pressure sensor switch 19 is turned ON, power from the battery 4 is supplied to the heater in the smoking device main body 5 by the power supply means 17d, and a smoking cartridge mounted to a steam chamber (not shown) inside is heated. When the smoking cartridge is heated by the heater, a steam containing nicotine, etc. is generated. The smoking cartridge is a throwaway substitute of an electronic cigarette containing tobacco leaf, and generates a nicotine containing steam through heating. Apart from this, there is one generating a steam containing nicotine, etc. with aroma through heating, and one containing no nicotine but an aromatic agent, generating a steam with aroma through heating.

The steam containing nicotine, etc. generated in the smoking device main body 5 is discharged into the mouth through suction at the nozzle portion 8. At this time, due to the negative pressure generated through suction, hydrogen emitted from the attachment 14 flows through the lid member 2, passes the periphery of the upper portion of the smoking device main body 5 exposed in the lid member 2 and the gap between itself and the inner wall of the nozzle portion 8, and is mixed with nicotine containing air before being guided into the mouth. It may also be possible to generate a steam containing nicotine, etc. with aroma by heating this.

FIG. 2 shows a specific structure example of the present smoking/hydrogen-suction device 1 in the state in which the smoking device main body 5 is held in it. FIG. 2(a) is a front view of the smoking/hydrogen-suction device 1, FIG. 2(b) is a plan view thereof, FIG. 2(c) is a left side view thereof, and FIG. 2(d) is a right side view thereof. FIG. 2(a) shows a state in which the lid member 2 has been removed from the smoking/hydrogen-suction device 1. In the state in which the lid member 2 has been removed (opened), there is provided a tubular smoking device receiving portion (hereinafter also referred to as the receiving portion) 20 extending downwards from the opening on the right side of the upper portion. The smoking device main body 5 is inserted into this receiving portion 20. The smoking device main body 5 is the main body portion of a general-purpose tubular heating-type electronic cigarette. In the following, an example of the general-purpose heating-type electronic cigarette will be described.

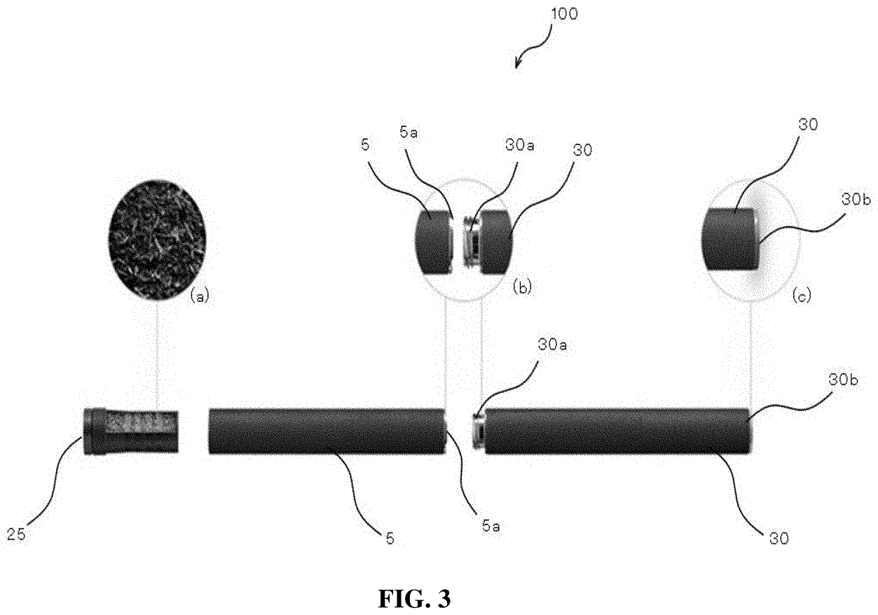

FIG. 3 schematically shows a heating-type electronic cigarette 100. The general-purpose heating-type electronic cigarette 100 shown in FIG. 3 is generally composed of a smoking cartridge 25 on the left-hand side, the smoking device main body 5, and a battery 30, and these members are connected in the longitudinal direction. The smoking cartridge 25 is a cylindrical throwaway member in which tobacco leaf in a granular state or the like is sealed in wrapping paper and which is provided with a hole for emitting steam. The tobacco leaf may be mixed with an aromatic agent until a uniform viscosity is attained, or for the purpose of promoting the health or quitting smoking, the cartridge may contain no tobacco leaf and be formed solely of an aromatic material (See the photographic view of FIG. 3(a)).

When smoking is to be performed, the cartridge 25 is pushed in from the upper end (the left end of FIG. 3) of the smoking device main body 5, and inserted into a hollow steam chamber (not shown). The inner wall of the upper portion of the substantially cylindrical smoking device main body 5 is formed in an upwardly divergent tapered configuration so that the smoking cartridge 25 may be easily inserted, or an insertion hole corresponding to the hollow surface of the steam chamber is provided at the bottom portion of the smoking cartridge 25 to facilitate fixation, enlarge the contact area, and facilitate heat conduction. When the suction negative pressure is generated in the upper portion of the smoking device main body 5, the device is placed in the ON state, and power is supplied from the rechargeable battery of a battery 30 described below, and the steam chamber is heated by the heater. Then, the water in the tobacco leaf in the smoking cartridge 25 is turned into steam, and at the same time, the nicotine ingredient (and/or the aromatic ingredient) is emitted. In this sense, as the device for taking in a steam containing nicotine, etc. via the mouth through suction, the smoking device main body 5 and the smoking cartridge 25 suffice. In the example of FIG. 3, the smoking device main body 5 is a semi-throwaway device in which the smoking cartridge 25 can be used five times.

The lower end (the right end in FIG. 3) of the smoking device main body 5 can be connected to the battery 30. In their connected state, the battery 30 and the smoking device main body 5 constitute one cylindrical bar. As shown in the example photograph of FIG. 3(b), at the lower end of the smoking device main body 5 and at the upper end of the battery 30, there are respectively provided attachments 5a and 30b that can be connected to each other. In the example of FIG. 3, a recess provided with a threaded groove is formed at the attachment 5a on the smoking device main body 5 side is formed, and a protrusion provided with a screw thread is formed at the attachment 30a on the battery 30 side, with the smoking device main body 5 and the battery 30 being connected to each other through axial rotational insertion.

Further, the attachment 5a also functions as a terminal electrically connected to the heater in the smoking device main body 5, and the attachment 30a also functions as a terminal electrically connected to the rechargeable battery in the battery 30. Thus, when the attachments 5a and 30a are connected together, power is supplied to the heater in the smoking device main body 5 from the rechargeable battery in the battery 30, and the tobacco leaf in the smoking cartridge 25 is heated via the steam chamber. Further, when negative pressure is exerted on the smoking device main body 5 through suction at the upper end thereof, the power from the battery 30 is supplied to the inner heater. At the same time, when the power from the battery 30 is being supplied, an LED 30b at the lower end of the battery 30 is ON.

Here, referring back to FIG. 2, the present smoking/hydrogen-suction device 1 will be described. The smoking device main body 5 of the general-purpose heating-type electronic cigarette of FIG. 3 described above is inserted into the receiving portion 20 of the smoking/hydrogen-suction device 1. The pressure sensor switch 19 is arranged at the bottom portion of the receiving portion 20, and at the upper end thereof, there is provided as an electrical terminal a protruding screw 19a of the same configuration as the attachment 30a. When the pressure sensor switch 19 is depressed, the power from the rechargeable battery (lithium battery) 4 is supplied to the protruding screw 19a. In this sense, the rechargeable battery 4 functions as a substitute for the battery 30 shown in FIG. 3. That is, in the state in which the smoking device main body 5 is held in the receiving portion 20 and in which the attachment 5a has been fastened to the protruding screw 19a, when the smoking device main body 5 is depressed from above, the power of the rechargeable battery 4 is supplied to the smoking device main body 5 via the protruding screw 19a, and the smoking cartridge 25 is heated, making it possible to inhale a steam containing nicotine, etc.

On the right side portion of the present smoking/hydrogen-suction device 1 (See FIG. 2(c)), there are provided an electronic cigarette ON/OFF switch 16c, an LED indicator 16b, and a main-power-source/hydrogen button 16a. The electronic cigarette ON/OFF switch 16c is the ON/OFF switch of the pressure sensor switch 19. When it is ON, when the attachment 5a at the lower end of the smoking device main body 5 is connected to the protruding screw 19a and depressed, power supply from the rechargeable battery 4 to the smoking device main body 5 is effected. When it is OFF, even when the pressure sensor switch 19 is depressed, no power supply from the rechargeable battery 4 is effected. The main-power-source/hydrogen button 16a is a button type power supply switch for the positive/negative electrodes 6 and 7 in the electrolysis tank 3 and the main power source. In accordance with the way it is depressed and the length of time it is depressed, it serves both as the ON/OFF switch for the main power source and as the ON/OFF switch for the power supply to the positive/negative electrodes 6 and 7.

In this example, when the main-power-source/hydrogen button 16a is kept depressed for three seconds, the positive and negative electrodes 6 and 7 are electrically conducted for five minutes to generate hydrogen, and when it is depressed intermittently three times in two seconds, the main power source is turned OFF. If the operation of turning OFF the main power source is not conducted, the switch is automatically turned OFF when 20 minutes elapses. While hydrogen is being generated, the main-power-source/hydrogen button 16a is lighted, and the residual power of the rechargeable battery 4 is indicated according to the lighting color. In this example, in the case where the residual power amount is 20 to 80%, it is lighted in blue, and in the case where the residual power amount is 80 to 100%, it is lighted in white. The LED indicator 16b is provided with two upper and lower LEDs. The upper LED is lighted when the power supply to the positive/negative electrodes 6 and 7 in the electrolysis tank 3 is effected, and the lower LED is lighted when the pressure sensor switch 19 is turned ON and the smoking device main body 5 is electrically conducted. The lighting of the electronic cigarette ON/OFF switch 16c, the LED indicator 16b, and the main-power-source/hydrogen button 16a is controlled by an inner indicator base 26.

As described above, when the pressure sensor switch 19 is turned ON, the power from the rechargeable battery 4 is also supplied to the pair of positive/negative electrodes 6 and 7 by the control substrate 17. As shown in FIG. 2(c), the pair of positive/negative electrodes 6 and 7 may be arranged horizontally at the bottom of the interior of the electrolysis tank 3, and as shown in FIG. 5, they may be arranged vertically. The rechargeable battery 4 allows input of power from a USB terminal 16d at the side portion of the smoking/hydrogen-suction device 1, and is thus charged (See FIG. 2(d)).

Next, referring to FIG. 4, to be described will be the inner structure of the electrolysis tank 3 and how electrolysis takes place in the electrolysis tank 3 in the case where the positive/negative electrodes 6 and 7 are electrically conducted. As shown in FIG. 4, the electrolysis tank 3 storing water is generally composed of a hollow tube member 3b extending in the longitudinal direction, a bottom member 3a closing the bottom portion of the tube member 3b, and lid members 3c and 3d closing the upper portion of the tube member 3a (The members 3c and 3d may be integrally molded). When the positive/negative electrodes 6 and 7 are electrically conducted, oxygen (O₂) is generated in the vicinity of the positive electrode 6, and hydrogen (H₂) is generated in the vicinity of the negative electrode 7. The oxygen and hydrogen generated are of lower specific gravity than water, so that they move upwards, each toward the gap 3g. Here, the electrolysis tank 3 is provided with a partition member 58 extending downwards from the upper end thereof and dividing the electrolysis tank 3 into a hydrogen gas generation layer 12 on the negative electrode 7 side and an oxygen gas generation layer 13 on the positive electrode 6 side. The lower end of the partition member 58 is provided with the gap 3g between itself and the upper surface of the bottom member 3a so as to fluidically connect the hydrogen gas generation layer 12 and the oxygen gas generation layer 13 to each other.

Due to this partition member 58, during the upward movement of the oxygen and hydrogen, the mixing of oxygen and hydrogen in the electrolysis tank 3 is hindered. On the other hand, in the lower portion of the gap 3g provided below the partition member 58 and not partitioned by the partition member 58, free movement of water (H₂O), that is, movement of ions (OH⁻) and (H⁺) necessary for generation of oxygen and hydrogen is possible. In this way, due to the partition member 58, the hindering of the mixing of oxygen and hydrogen is attained while performing electrolysis.

The lid member 3c closes the upper portion of the oxygen gas generation layer 13. An opening 3e is provided between a part of the lid member 3c or the lid member 3c and the partition member 58 or the tube member 3b. The opening 3e is closed by an oxygen transmission membrane 9. Thus, even if hydrogen is allowed to leak from the hydrogen gas generation layer 12 to the oxygen gas generation layer 13 due to the gap 3g, etc., the gas emitted to outside due to the oxygen transmission membrane 9 is restricted to oxygen. This oxygen transmission membrane 9 may be arranged at an electrolyte injection port/hydrogen generation port (described below) shown in FIG. 2. However, it is desirable for the membrane to be arranged at the hole dedicatedly provided in the electrolysis tank 3.

Further, also in the hydrogen gas generation layer 12, the upper portion of the hydrogen gas generation layer 12 is closed by the lid member 3d. However, an opening 3f is provided in the upper portion of the tube member 3b on the hydrogen gas generation layer 12 side. The opening 3f is connected to a bypass channel 3h. Thus, the hydrogen in the hydrogen gas generation layer 12 generated at the negative electrode 7 flows into the bypass channel 3h, and flows upwards.

Regarding the hydrogen channel from the opening 3f to the bypass channel 3h of FIG. 4, in the example of FIG. 2, the electrolyte injection port/hydrogen generation port 14 corresponds to the opening 3f, and the gap between the upper portion of the electrolysis tank 3 and the lid member 2 corresponds to the bypass channel 3. As described above, the electrolyte injection port/hydrogen generation port 14 functions as an inflow port through which the electrolyte or water is injected into the electrolysis tank 3 and functions as the opening 3f emitting the hydrogen in the electrolysis tank 3 to outside. It is of a configuration that can be detachably fastened to the electrolyte injection port/hydrogen generation port 14 by a screw. In the state in which it has been removed by loosening the screw, the electrolyte or water is injected from the opening 3f. In the state in which the screw has been fastened, leakage of the electrolyte, etc. in the electrolysis tank 3 is suppressed, whereas hydrogen is emitted from a hydrogen, etc. transmission membrane (not shown) closing a hole or the like separately provided at the electrolyte injection port/hydrogen generation port 14.

As indicated by the dotted line of FIG. 2, the emitted hydrogen flows to the left (in the direction of the smoking device main body 5) through the lid member 2. The lid member 2 has a tubular nozzle portion 8 protruding, with an opening being provided at the left end and the upper end. It is a detachable integral member which covers the upper end of the smoking device main body 5 and the electrolyte injection port/hydrogen generation port 14 and which is arranged at the upper portion of the electrolysis tank 3. When the lid member 2 is attached to the upper portion of the electrolysis tank 3, the nozzle portion 8 is in a state in which the upper end of the smoking device main body 5 is inserted into the opening in a telescopic fashion, with a gap 26 being provided in the periphery. When the nozzle portion 8 is sucked at, the hydrogen (high concentration hydrogen containing air) having come flowing from the electrolyte injection port/hydrogen generation port 14 ascends through the gap 26 to be discharged to outside (See the dotted line in FIG. 2). As a result, it is possible to take in high concentration hydrogen. In the case where power supply is effected to the smoking device main body 5, it is possible to take in a mixture gas consisting of the air containing nicotine and/or aromatic agent from the smoking cartridge 25 and high concentration hydrogen. The present smoking/hydrogen-suction device 1 is equipped with a cover 1a which is openable and closable. In the example of FIG. 2, the cover 1a is open. In order that the liquid amount inside the electrolysis tank 3 may be visually checked, there is provided at the side portion of the smoking/hydrogen-suction device 1, an opening (an electrolyte checking window) 3i through which it is possible to visually check the liquid amount in the electrolysis tank 3.

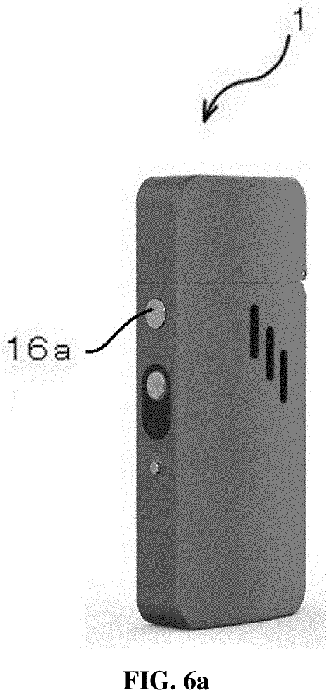

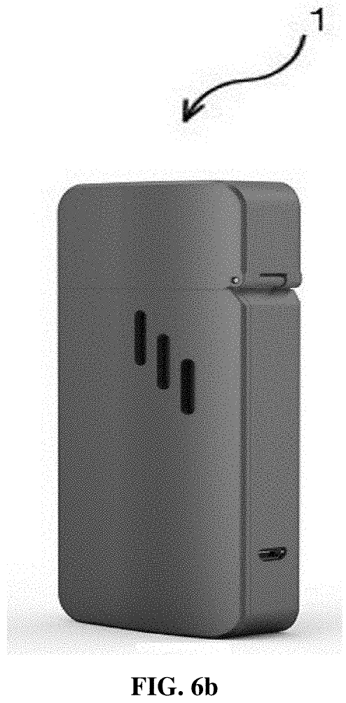

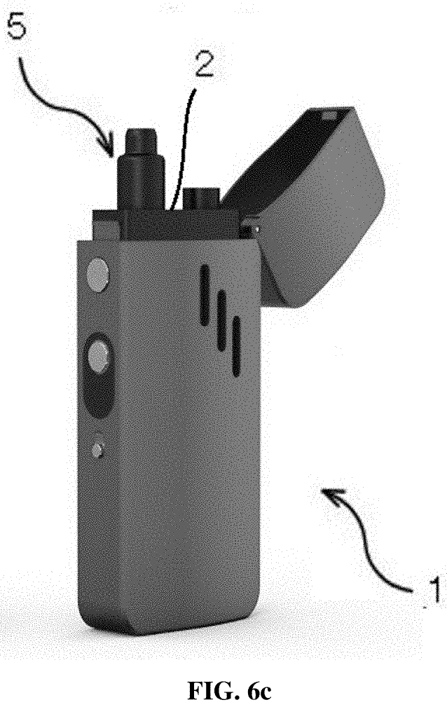

For reference, FIG. 6 shows an external photographic view of the smoking/hydrogen-suction device 1 of the embodiment of the present invention. FIG. 6(a) shows the smoking/hydrogen-suction device 1 as seen obliquely from the left front side, FIG. 6(b) shows the smoking/hydrogen-suction device 1 as seen obliquely from the right front side, and FIG. 6(c) shows the smoking/hydrogen-suction device 1 as seen obliquely from the left front side with the cover 1a being open. In terms of design, the example has some portions different from those of the example of FIG. 2. However, the main structure is substantially the same, and the same reference numerals as those of FIG. 2 are used in FIG. 6.

The present invention is not restricted to the smoking/hydrogen-suction device according to the embodiment described above. It will be understood by those skilled in the art that other modifications and improvements are possible without departing from the scope of the claims and of the gist and teachings of the specification, etc.

### REFERENCE SIGNS LIST

1 smoking/hydrogen-suction device
2 lid member
3 electrolysis tank
4 rechargeable battery
5 smoking device main body
6 positive electrode
7 negative electrode
8 nozzle portion
17 control means
18 operation button (operation means)
19 pressure sensor switch
19a protruding screw
20 smoking device receiving portion (receiving portion)
25 smoking cartridge

1. A smoking/hydrogen-suction device which detachably accommodates a tubular portable heating-type smoking device main body and which makes it possible to smoke and suck in hydrogen, comprising:
a rechargeable battery arranged in a battery receiving portion;
a control substrate controlling power supply from the battery;
a pair of positive/negative electrodes that are electrically conducted to or cut off from a positive electrode and a negative electrode of the battery by the control substrate;
an electrolysis tank which is capable of storing water, into a lower portion of which the pair of positive/negative electrodes are inserted, and which has an injection port for an electrolyte or water and a hydrogen generation port fluidically connected to an air layer in the electrolysis tank; and
a smoking device receiving portion having a tubular hole into which the smoking device main body can be inserted from above and having at its bottom portion a terminal that can be electrically connected to a terminal at a lower portion of the smoking device,
wherein when power supply from the battery to the smoking device main body is effected by the control substrate, a smoking cartridge mounted to an upper portion of the smoking device is heated to generate a nicotine or aroma containing steam from the upper portion;
when power supply from the battery to the pair of positive/negative electrodes is effected by the control substrate, the electrolysis tank generates hydrogen from the positive electrode, and the hydrogen is emitted from the hydrogen generation port; and
the smoking/hydrogen-suction device further comprises a lid member covering the hydrogen generation port of the electrolysis tank and the smoking cartridge and guiding the nicotine or aroma containing steam and the hydrogen respectively emitted from the smoking cartridge and the hydrogen generation port to a nozzle portion having a through hole.

2. The smoking/hydrogen-suction device according to claim 1, wherein the water injection port of the electrolysis tank is openable and closable, and the hydrogen generation port is shielded with a material solely allowing transmission of gas and not allowing transmission of liquid in the electrolysis tank, the water injection port and the hydrogen generation port being formed as an integral attachment.

3. The smoking/hydrogen-suction device according to claim 2, wherein in the state in which the smoking device main body is held in the smoking device receiving portion, the upper end of the smoking device main body and the attachment of the electrolysis tank are arranged in parallel, and the nozzle portion of the lid member covers the upper end of the smoking device in a telescopic fashion, the hydrogen guided from the hydrogen generation port of the attachment within the lid member being emitted to outside from the gap between the nozzle portion and the upper end of the smoking device.

4. The smoking/hydrogen-suction device according to any one of claim 1, wherein when power supply is effected from the battery to the smoking device main body by the control substrate, the heater in the smoking device main body is heated, and the smoking cartridge containing a wet smokable substance is heated.

5. The smoking/hydrogen-suction device according to any one of claim 1, further comprising, at the bottom portion of the smoking device receiving portion, a pressure sensor switch supplying power from the battery to the smoking device main body by the control substrate when the smoking device main body is inserted and downwardly depressed.

6. The smoking/hydrogen-suction device according to any one of claim 1, wherein the control substrate receives an electrical conduction signal to the pair of positive/negative electrodes based on an operation signal from the user to perform power supply from the battery to the pair of positive/negative electrodes based on each signal, and receives an electrical conduction signal to the smoking device main body based on a power supply signal from the pressure sensor switch to effect power supply from the battery to the smoking device main body based on the signal.

7. The smoking/hydrogen-suction device according to claim 6, further comprising an ON/OFF switch of the pressure sensor switch.
